SEQUENCE LISTING (1 sequence)

SEQ ID NO: 1 - protein, 12 aa, Tenebrio molitor
  MISC_FEATURE (3)..(3): X=any amino acid
CTXSXXCXXAXT

(19) United States
(12) Patent Application Publication
(10) Pub. No.: US 2005/0161631 A1
(43) Pub. Date: Jul. 28, 2005

(54) Antifreeze proteins for inhibition of clathrate hydrate formation and reformation

(76) Inventors: Virginia K Walker, Ontario (CA); John A Ripmeester, Ontario (CA); Huang Zeng, Ontario (CA)

Correspondence Address: LICATLA & TYRRELL P.C., 66 E. MAIN STREET, MARLTON, NJ, 08053, US

(21) Appl. No.: 10/510,557

(22) PCT Filed: Apr. 11, 2003
(86) PCT No.: PCT/CA03/00528

Related U.S. Application Data
(60) Provisional application No. 60/372,522, filed on Apr. 12, 2002.

(51) Int. Cl. C09K 3/18; C07K 14/47

(52) U.S. Cl. 252/70; 530/395; 536/23.1

(57) ABSTRACT

Antifreeze proteins, active fragments of these antifreeze proteins and mimetics thereof for use in inhibiting clathrate hydrate formation and reformation and designing and selecting inhibitors of clathrate hydrate formation and reformation are provided.

This application claims the benefit of provisional U.S. Application Ser. No. 60/372,522, filed Apr. 12, 2002, which is herein incorporated by reference in its entirety.

### FIELD OF THE INVENTION

The present invention provides antifreeze proteins and active fragments thereof, as well as mimetics of these antifreeze proteins and active fragments thereof, for use in inhibiting formation of clathrate hydrates and decreasing the rate at which clathrate hydrates reform following melting. Preferred antifreeze proteins or active fragments thereof useful in the present invention are those proteins or fragments containing or forming a β-helix or β-helices, a β-roll, a glycoprotein or a globular structure. Such antifreeze proteins may be derived, for example, from animals, plants, fungi, protists and bacteria. The antifreeze proteins, active fragments of the proteins and mimetics of the antifreeze proteins or active fragments thereof are useful in preventing blocking or plugging resulting from gas hydrate formation in conduits and machinery of, for example, drilling and exploration operations and pipe-line transport operations of natural oil and gas, bacterial fermentation processes, and disposal of carbon dioxide wastes.

### BACKGROUND OF THE INVENTION

Thermal hysteresis refers to the ability to produce a difference between the freezing and melting temperatures of a solution. By definition, the equilibrium melting point and freezing points of water are identical. However, thermal hysteresis proteins, identified in some species of fish, insects, plants, fungi and bacteria have the ability to lower the non-equilibrium freezing point of water without lowering the melting point (equilibrium freezing point). Thus, when thermal hysteresis proteins are added to a solution, they produce a difference between the freezing point and melting temperatures of the solution.

In the absence of a thermal hysteresis protein, a small ice crystal of approximately 0.25 mm in diameter that is about to melt at the melting point temperature will normally grow noticeably if the temperature is lowered by 0.01° to 0.02° C. However, in the presence of a thermal hysteresis protein, the temperature may be lowered by 1° to 6° C. below the melting point, depending upon the specific activity of the protein, the size of the initial ice crystal, the rate of temperature reduction, and the concentration of the protein present, before noticeable ice crystals form. Because of their ability to lower the freezing point of aqueous solutions, these thermal hysteresis proteins are commonly referred to as antifreeze proteins. However, unlike antifreezes such as glycerol, antifreeze proteins lower the freezing point of aqueous solutions via a non-colligative mechanism that does not depress the vapor pressure or raise the osmotic pressure of water.

Antifreeze proteins were first discovered and have been best studied in marine teleost fish inhabiting seas wherein subzero temperatures occur. The serum of these fish would be expected to freeze at −0.7° C. but because of the presence of antifreeze proteins, ice crystals in the serum fail to grow in size until the temperature has been lowered to −1.8° C. This fish protein is rich in alanines (approximately 65 mol %) and is predominantly a single α-helix. Three other types of antifreeze proteins have also been isolated from different cold-water fish.

Antifreeze proteins have also been identified in many insects and other terrestrial arthropods including certain spiders, mites and centipedes. Antifreeze proteins have been isolated from four species of insects including Tenebrio molitor, Oncopeltus fasciatus, Choristoneura fumiferana and Dendroides canadensis. In contrast to fish antifreeze proteins, insect antifreeze proteins are rich in cysteine residues (U.S. Pat. No. 5,627,051). Further, Tenebrio molitor antifreeze proteins contain tandem 12 residue repeats which translate into an exceptionally regular β-helix (Liou et al. Nature 2000 406: 322-328; WO 99/00493). Insect antifreeze proteins are much more effective than fish antifreeze proteins at depressing freezing points by ice-growth inhibition (Tyshenko et al. Nature Biotechnol. 1997 15: 887-890; Graham et al. Nature 1997 388: 727-728).

Nucleic acid sequences encoding Dendroides canadensis antifreeze proteins are disclosed in U.S. Pat. No. 5,627,051.

Nucleic acid sequences encoding Choristoneura fumiferana antifreeze proteins are disclosed in U.S. Pat. No. 6,008,016.

Nucleic acid sequences encoding Tenebrio molitor antifreeze proteins are disclosed in WO 99/00493 and U.S. Pat. No. 6,392,024.

Low levels of thermal hysteresis activity are also common in overwintering plants and certain fungi and bacteria.

Antifreeze proteins are believed to exert their effect by adsorbing onto the surface of potential seed crystals via hydrogen bonding, thus interfering with the addition of water molecules to the crystal and further growth of the crystal (Yeh and Feeney Chem. Rev. 1996 96: 601-617; Davies et al. Curr. Opin. Struct. Biol. 1997 7: 828-834; Harrison et al. Nature 1987 328: 241; DeOliveira et al. J. Am. Chem. Soc. 1997 119: 10627-10631)). Thus, in the presence of these proteins, the growth of the crystal is forced into many highly curved fronts with high surface free energy (WO 99/00493). Antifreeze proteins also inhibit the recrystallization of ice, a phenomenon wherein grains of small polycrystalline ice change size and shape when held for a prolonged period of time at a sub-melting temperature (Knight et al. Nature 1984 308: 295-296).

Antifreeze proteins derived from insects have been disclosed as being useful in enhancing the supercooling properties of a fluid to prevent the freezing of fluids at temperatures below their equilibrium melting temperature, to prevent or limit ice growth or recrystallization of frozen goods, and provide protection from damage that normally results from freezing biological materials. See U.S. Pat. No. 5,627,051, U.S. Pat. No. 6,008,016, U.S. Pat. No. 6,348,569, U.S. Pat. No. 6,392,024 and WO 99/00493.

Clathrate hydrates are inclusion compounds based on three-dimensional ice-like frameworks made up of hydrogen-bonded water molecules. Each framework structure has a number of polyhedral cavities of various dimensions that house guest molecules. To date, three families of clathrate hydrates have been determined: body-centered cubic structure I, diamond lattice cubic structure II (Jeffrey, G. A. in Inclusion Compounds Vol. 1, 135-190 (Academic, London 1984)), and hexagonal structure H (Ripmeester et al. Nature 1987 325: 135-136). Because they form from stacked layers of pentagonal dodecahedra, structure II and structure H have a common structural motif, and it is possible that intergrowths of these structures may occur as well. In a natural environment, guest molecules trapped in hydrates typically are hydrocarbon gases such as methane, ethane, propane, butane and isobutane. Potentially, alkenes, alkynes, methyl substituted butanes and pentanes, cyclic alkanes from cyclopropane to cyclooctane, cycloalkenes and their methyl-substituted analogs can be present as guests as well. Other possible guests in natural hydrates are small molecules such as CO₂, H₂S, the noble gases (Ar, Kr, Xe), oxygen and nitrogen. Such clathrate hydrates can also be formed in laboratory and industrial settings as well as clathrate hydrates formed by ethers, ketones, aldehydes, mercaptans, sulfides, halogenated hydrocarbons, and a number of inorganic molecules including, but not limited to, SF₆, PH₃, H₂Se, SO₂, ClO₂, CO, ClO₃F, SO₂F₂, NF₃, Cl₂, Br₂, and COS.

The formation of clathrate hydrates is a serious, billion dollar per year problem in the production and transportation of hydrocarbons in the oil and gas industry. Gas hydrate plug formation poses a danger in both drilling and exploration operations and in pipe-line transport operations as these hydrates cause blocking or plugging of the pipelines (Sloan, E. D. Jr. Clathrate Hydrate of Natural Gases, second edition, Marcel Dekker, Inc., New York, USA (1998)). Commonly employed methods to counteract hydrate formation include gas dehydration, thermal insulation, pressure reduction, and addition of antifreeze reagents in the form of methanol or ethylene glycol.

Synthetic polymers have been investigated for use as inhibitors for clathrate hydrate. Most promising as a synthetic polymer inhibitor is polyvinylpyrrolidone (PVP) and polymers related thereto that have a long polyethylene backbone with an amide linkage in the side groups (Lederhos et al. Chem. Eng. Sci. 1996 51: 1221; Englezos, P. Chem. Eng. Res. Des. 1992 70: 43-47). The synthetic polymers are often referred to as kinetic inhibitors. They appear to cover the surface of clathrate hydrate crystals thus slowing down the growth of the crystals.

Another class of inhibitors is based on quaternary ammonium salts with several long alkyl or alkyl ester chains that are effective as anti-agglomerants.

Despite high costs, injection of methanol or ethylene glycol antifreeze reagents to inhibit gas hydrate formation is still used most extensively (Sloan, E. D. Gas Research Institute Topical Report GRI-91/0302; Gas Research Institute: Chicago Ill., Jun. 1, 1992). Currently, this involves a routine calculation based on estimate of water content. A certain quantity of methanol or ethylene glycol is added accordingly.

Theoretical calculations (Edwards et al. Annals of the NY Acad. Sci. 1994 715: 543) supported a hypothesis for using the fish thermal hysteresis protein, AFP Type I to inhibit hydrate formation. However, early experiments gave conflicting results (Lederhos et al. Chem. Eng. Sci. 1996 51: 1221). In particular, Type I fish AFP was reported to “not offer a practical solution to holding hydrates” (http://www.oilonline.com/new/features/oe/20010201.holding_{—}0.25.asp).

Formation of CO₂ clathrates is also envisioned in disposal of CO₂ containing wastes (www.netl.doe.gov/publications/factsheets/program/prog019.pdf).

The present invention provides methods for inhibiting clathrate hydrate formation and decreasing the rate of clathrate hydrate reformation using an antifreeze protein, an active fragment of the antifreeze protein, or a mimetic of the antifreeze protein or active fragment thereof.

### SUMMARY OF THE INVENTION

In one aspect, the present invention relates to a method for inhibiting clathrate hydrate formation using an antifreeze protein, an active fragment of the antifreeze protein, or a mimetic of the antifreeze protein or active fragment thereof.

Another aspect of the present invention relates to a method for decreasing the rate at which clathrate hydrates reform and/or decreasing the rate of nucleation following a freeze-thaw cycle using an antifreeze protein, an active fragment of the antifreeze protein or a mimetic of the antifreeze protein or active fragment thereof. Preferred in this method is addition of a second, different clathrate hydrate inhibitor.

Another aspect of the present invention relates to a method for designing and selecting hydrate inhibitors by comparing the ability of a test compound to inhibit clathrate formation and/or reformation to the ability of an antifreeze protein or an active fragment of the antifreeze protein to inhibit clathrate formation and/or reformation.

Yet another aspect of the present invention relates to a composition for inhibiting clathrate hydrate formation and/or decreasing the rate of reformation of clathrate hydrate and/or decreasing the rate of nucleation following a freeze-thaw cycle which comprises an antifreeze protein, an active fragment of an antifreeze protein, or a mimetic of the antifreeze protein or active fragment thereof in an aqueous solution. In a preferred embodiment, the composition further comprises a second, different clathrate hydrate inhibitor.

In a preferred embodiment of the present invention, the antifreeze protein contains or forms a β-helix or β-helices, a β-roll, a glycoprotein or a globular structure. Such preferred antifreeze proteins can be derived from animals, plants, fungi, protists and bacteria.

### DETAILED DESCRIPTION OF THE INVENTION

The present invention provides methods and compositions for inhibiting clathrate hydrate formation and decreasing the rate at which clathrate hydrates reform. The methods and compositions of the present invention are particularly useful in preventing blocking or plugging resulting from gas hydrate formation in conduits and machinery that are used, for example, in drilling and exploration operations and in pipe-line transport operations of natural oil and gas, bacterial fermentation procedures, and disposal of CO₂ waste products. Compositions used in the methods of the present invention comprise an antifreeze protein, preferably an antifreeze protein containing or forming a β-helix or β-helices, a β-roll, a glycoprotein or a globular structure, an active fragment of the antifreeze protein or a mimetic thereof. By “containing or forming a β-helix or β-helices, a β-roll, a glycoprotein or a globular structure” it is meant that the protein either comprises one or more β-helices, β-rolls, glycoproteins or globular structures in its natural structure or forms one or more β-helices, β-rolls, glycoproteins or globular structures under selected conditions. Also preferred are antifreeze proteins containing or forming a repeating sequence or structure. Examples of antifreeze proteins that contain or form a β-helix or β-helices, a β-roll, a glycoprotein or a globular structure and/or a repeating sequence or structure include antifreeze proteins of animals, plants, fungi, protists and bacteria. Most preferred for use in the present invention are those antifreeze proteins derived from either insects or plants. Antifreeze proteins, active fragments and mimetics thereof used in the present invention are environmentally benign. Accordingly, loss and/or recovery is not a concern. In addition, the antifreeze proteins, active fragments and mimetics thereof remain in the aqueous phase, and thus do not contaminate valuable products such as in the oil drilling, exploration and transport operations.

Examples of insect antifreeze proteins useful in the present invention include, but are not limited to: antifreeze proteins derived from the mealworm beetle Tenebrio molitor as described in WO 99/00493, the teachings of which are herein incorporated by reference in their entirety; antifreeze proteins derived from the spruce budworm Choristoneura fumiferana as described in U.S. Pat. No. 6,008,016, the teachings of which are herein incorporated by reference in their entirety; antifreeze proteins derived from the milkweed bug Oncopeltus fasciatus (Patterson et al. J. Comparative Physiol. 1981 142: 539-542); and antifreeze proteins derived from Dendroides canadensis as described in U.S. Pat. No. 5,627,051, the teachings of which are herein incorporated by reference in their entirety. In a preferred embodiment, the antifreeze protein, active fragment thereof, or mimetic of the antifreeze protein or active fragment thereof is derived from either the mealworm beetle Tenebrio molitor or the spruce budworm Choristoneura fumiferana.

cDNAs encoding antifreeze proteins of Tenebrio molitor have been isolated and found to encode 7 to 13 kDa cysteine rich proteins, composed largely of 12 amino acid repeats (Graham et al. Nature 1997 388: 727; WO 99/00493). Difference in size range of these proteins is largely due to differences in the number of amino acid repeats in the proteins. The cysteines of the amino acid repeats are important in stabilizing the structure via disulfide bridges believed to allow for formation of a β-helix or β-helices in the correct position so that the repeats form hydrogen bonds with the ice lattice (Liou et al. Nature 2000 406: 322-328).

cDNAs of Choristoneura fumiferana encoding antifreeze proteins of approximately 9 kDa and 12 kDa have also been cloned (Doucet et al. Eur. J. Biochem 2002 269: 38-46). These antifreeze proteins are also rich in cysteines necessary for disulfide bonding and formation of a β-helix or β-helices, Further, five conserved threonine button (Thr-Xaa-Thr) ice binding motifs have been identified in the protein (Doucet et al. Eur. J. Biochem 2002 269: 38-46).

Threonines of the threonine buttons match the ice lattice in antifreeze protein/ice models. In some antifreeze proteins useful in the present invention, threonines are substituted by valine or isoleucine, amino acids with methyl R groups and similar spatial volumes to threonine, thus indicating that non-polar interactions may be important for the inhibition of ice growth. These properties may also be indicative of insect antifreeze proteins interacting with the hydrate surfaces and adsorbing to clathrates composed of various crystal morphologies.

Insect antifreeze proteins exhibit hyperactivity as compared to fish antifreeze proteins in thermal hysteresis assays; their activity is 1-2 orders of magnitude higher, on a molar basis, than fish antifreeze proteins isolated.

Examples of plant antifreeze proteins which may be useful in the present invention include, but are not limited to, those derived from Lolium perenne (Lm) rye grass (Kuiper et al. Biophysical J. 2001 81: 3560-3566) Solanum dulcamara bittersweet nightshade, Secala cereale winter rye and Daucus carota carrot (Worrall et al. Science 2; 282 (5836): 115-5). Also see U.S. Pat. No. 5,972,679, U.S. Pat. No. 6,162,789, U.S. Pat. No. 6,156,880, U.S. Pat. No. 6,090,917, and U.S. Pat. No. 6,096,867.

Additional antifreeze proteins for use in the present invention can be routinely selected based upon the structure of the antifreeze protein. Structural characteristics of an antifreeze protein indicative of its utility in inhibiting clathrate hydrate formation and/or decreasing the rate of clathrate hydrate reformation include, for example, a repetitive amino acid sequence rich in cysteines for disulfide bonding, the presence of one or more threonine button (Thr-Xaa-Thr) ice binding motifs, and/or one or more β-helices formed by the repetitive amino acid sequences.

For purposes of the present invention, by “derived from” it is meant to include antifreeze proteins, fragments or mimetics which originated from a particular species and were isolated from that particular species as well as polypeptides identical in amino acid sequence which are recombinantly expressed in a host cell expression system or chemically synthesized. It is also meant to be inclusive of derivatives of these antifreeze proteins, fragments or mimetics. By “derivative” it is meant a polypeptide or fragment thereof that is substantially similar in primary structural sequence to an antifreeze protein or fragment described herein but which includes chemical and/or biochemical modifications that are not found in the native polypeptide. An example of such a modification is the addition of a label such as a radioactive isotope, a fluorophore or an enzymatic label useful in tracing the antifreeze protein or active fragment thereof during use and in isolating the antifreeze protein or active fragment thereof after use for recovery and/or recycling. Additional exemplary modifications which can be made include those to enhance stability such as formation of salt bridges.

By “fragment” as used herein it is meant any amino acid sequence shorter than the full length antifreeze protein but which maintains similar activity to the full length antifreeze proteins. Fragments of the present invention preferably may comprise a single contiguous sequence identical to a portion of the antifreeze protein sequence. Alternatively, the fragment may comprise several different shorter segments, each segment identical in amino acid sequence to a different portion of the amino acid sequence of the antifreeze protein, but linked via amino acids differing in sequence from the antifreeze protein. An exemplary fragment of the present invention comprises one or more of the amino acid residue repeats reported in the mealworm beetle protein. The 12 amino acid repeat sequence of the mealworm beatle protein comprises cys-thr-xaa-ser-xaa-xaa-cys-xaa-xaa-ala-xaa-thr (SEQ ID NO:1). This 12-residue repeat translates into an exceptionally regular β-helix (Liou et al. Nature 2000 406: 322-328) believed to be responsible at least in part for the thermal hysteresis activity of these insect proteins. Accordingly, a fragment comprising one or more of these 12-residue repeats is expected also to exhibit thermal hysteresis activity. Another exemplary fragment comprises the threonine button identified as an ice binding motif in the spruce budworm.

For purposes of the present invention, by “similar activity”, “activity” or “active” when used with respect to fragments, it is meant that the fragment exhibits comparable or more preferably enhanced thermal hysteresis activity as compared to the full length antifreeze protein from which the fragment was derived at an equivalent concentration. The thermal hysteresis activity can be measured in the instant invention by various methods as exemplified herein. In a preferred embodiment, a fragment with similar activity will exhibit a comparable or enhanced ability to inhibit clathrate hydrate formation and/or decrease the rate at which clathrate hydrates reform.

By “mimetic” as used herein it is meant to be inclusive of polypeptides, which may be recombinant, and peptidomimetics, as well as small organic molecules which exhibit similar or enhanced thermal hysteresis activity as compared to the antifreeze proteins described herein.

As used herein, the term “peptidomimetic” is intended to include peptide analogs which serve as appropriate substitutes for an antifreeze protein or active fragment thereof in interactions with ice crystals. The peptidomimetic must possess not only affinity, but also efficacy and function. That is, a peptidomimetic exhibits function(s) of an antifreeze protein or active fragment thereof, without restriction of structure. Peptidomimetics of the present invention, i.e. analogs of antifreeze proteins or active fragments thereof which bind to ice crystals and inhibit clathrate hydrate formation and/or decrease the rate of clathrate hydrate reformation, include amino acid residues or other moieties which provide the functional characteristics described herein. Peptidomimetics and methods for their preparation and use are described in Morgan et al. 1989, “Approaches to the discovery of non-peptide ligands for peptide receptors and peptidases,” In Annual Reports in Medicinal Chemistry (Vuirick, F. J. ed), Academic Press, San Diego, Calif., 243-253.

Mimetics of the present invention may be designed to have a similar structural shape to the antifreeze proteins. For example, antifreeze proteins useful in the present invention have been identified as comprising an amino acid residue repeat that translates into an exceptionally regular β-helix (Liou et al. Nature 2000 406: 322-328), a β-roll, a glycoprotein and/or a globular structure. Mimetics of the present invention can be designed to have a structure which mimics this β-helix or β-helices, a β-roll, a glycoprotein and/or a globular structure. For polypeptide mimetics or peptidomimetics mimicking the β-helix structures, preferred amino acids for inclusion are Ala (A), Glu (E), Leu (L), Met (M) as these amino acids prefer a helix structure. In contrast amino acids Pro (P), Gly (G), Tyr (Y), and Ser (S) do not prefer a helix structure and thus are preferably not included in at least the β-helix forming portion of the mimetics. Mimetics can also be designed with extended and/or additional amino acid residue repeats as compared to naturally occurring antifreeze proteins. Host cells can be genetically engineered to express such mimetics in accordance with routine procedures. Alternatively, mimetics may be produced by chemical means. Antifreeze proteins used in the present invention have also been reported to comprise significant amounts of cysteines involved in disulfide bridges, which when reduced results in a complete loss of thermal hysteresis activity. Accordingly, mimetics of the present invention may also comprise compounds with high amounts of cysteines and/or disulfide bridges. A threonine button (Thr-Xaa-Thr) has also been identified in the spruce budworm as contributing the ice-binding capabilities. Mimetics of the present invention may also comprise a threonine button or threonine buttons.

Parameters that are used to assess the effectiveness of hydrate inhibitors include measurement of hydrate formation kinetics (rate of gas uptake), driving force measurements (e.g. the degree of sub-cooling required to initiate hydrate formation), and induction time (Montfort et al. Ann. N.Y. Acad. Sci. 2000 912: 753; Svartaas et al. Ann. N.Y. Acad. Sci. 2000 912: 744). Various methods for measuring these parameters can be used to assess the ability of an antifreeze protein, an active fragment thereof or a mimetic of the antifreeze protein or fragment thereof to inhibit clathrate hydrate formation.

For example, the induction time of tetrahydrofuran (THF) clathrate hydrate can be determined in the presence and absence of an antifreeze protein, an active fragment thereof or a mimetic. Determination of the induction time for THF clathrate hydrate formation in the presence or absence of antifreeze proteins is used as an assay for the inhibition activity of the antifreeze protein, an active fragment thereof or a mimetic. Induction time is the time required for the onset point of the crystallization. In this assay, samples are kept at constant temperature with constant stirring and a sudden rise of temperature signals the onset point of crystallization of THF clathrate hydrate. The effect of a number of parameters including concentration (typically ranging from 1.0 mg/ml to 0.2 mg/ml) of the antifreeze protein, an active fragment thereof or a mimetic, temperature (−1.0° C., 0.0° C., 1.0° C.), gas (ambient air/degassed at high vacuum), and salt (NaCl) are determined and the data subjected to statistical analysis. Freezing points, obtained with differential scanning calorimetry (DSC) are used as a measure of inhibition activity and for comparison with known kinetic inhibitors.

The growth rate of THF hydrate in the presence of an antifreeze protein, an active fragment thereof or a mimetic can also be determined and compared to activities of other antifreeze proteins and/or known kinetic inhibitors. Experiments are performed by capturing images of the THF hydrate crystals growing in solution, using a specially built crystal-growth-observation apparatus (Makogon et al. Crystal Growth 1997 179: 258). Effects of concentration of the antifreeze protein, active fragment thereof or mimetic, temperature, gas and salt on the growth rate can also be examined.

Growth inhibition experiments provide insight into face-selective THF hydrate crystal growth. When THF hydrates are grown in the absence of inhibitors, they predominantly display an octahedral structure. This can be perturbed in the presence of kinetic inhibitors where the growth habit is changed such that octahedral crystal faces are more dominant. The growth habit of the THF hydrate crystal should be related to the interaction between the antifreeze protein, the active fragment thereof or the mimetic, and specific crystal faces of THF hydrate. Of particular interest is the comparison of the morphology of THF, grown in the presence and absence of the antifreeze protein, the active fragment thereof or the mimetic. The growth kinetics of different faces are measured as a function of the different types of antifreeze proteins, active fragments thereof and mimetics, their concentrations and the incubation temperature.

Also of interest is examination of the interface between the THF hydrate and the antifreeze protein, the active fragment thereof or the mimetic. The concentration of antifreeze protein, active fragment thereof or the mimetic on the surface can be determined by THF activity assays or by assay of the inhibition of ice recrystallization of the solution melted from THF hydrate crystal grown from THF-H₂O solution with the antifreeze protein, the active fragment thereof or the mimetic.

The mechanism of inhibition can also be studied using various methods, examples of which are provided as follows.

Nucleation inhibition, defined as inhibition of the formation of crystal embryos, is examined via homogenous nucleation (the formation of a critical size embryo when the system only has molecules of the metastable substance) using one of several techniques. For example, an emulsion of THF-H₂O can be made with and without the antifreeze protein, active fragment thereof or the mimetic, in silicon oil. This emulsion can then be used to measure the nucleation temperature by DSC. The induction time can also be recorded.

Raman spectroscopy can also be used to assess the intra-molecular OH stretching in the sample of THF-H₂O in the absence and presence of the antifreeze protein, the active fragment or the mimetic thereof. Raman spectroscopy is the measurement of the wavelength and intensity of inelastically scattered light from molecules and is complementary to infrared absorption spectroscopy. Results from Raman spectroscopy are related to the de-structuring effect of the antifreeze protein, the active fragment thereof or the mimetic on the adjacent water molecules.

A miniature variable pressure and temperature cell was constructed for measuring Raman spectra of gas hydrate systems. This cell was built to be suitable at lower pressure ranges than cells used previously and is useful for mixed hydrates containing two or more guest molecules. This cell has been used successfully for optical microscopy applications on the THF system.

Nucleation inhibition can also be tested using heterogeneous nucleation, wherein crystal embryos form on the surface of some foreign material with which the metastable substance is in contact. In these assays, a nucleation agent such as AgI is used and the induction time and nucleation temperature is measured to evaluate the interaction between AgI and the antifreeze protein, the active fragment thereof or the mimetic.

The conformation of an antifreeze protein, active fragment thereof or a mimetic in solution can be studied by circular dichroism spectroscopy (CDS). This technique measures differences in the absorption of left-handed polarized light versus right-handed polarized light that arise due to structural asymmetry. It allows one to gain information about the secondary structure of proteins and polypeptides in solution. CDS is useful to investigate the conformation of antifreeze proteins, active fragments thereof and mimetics in solution at different temperatures. The effect of salt (as described for the induction time experiments) can also be studied.

The status of water around the antifreeze protein, the active fragment thereof or the mimetic can also be investigated. Water in contact with an antifreeze protein has properties different from bulk water in that it does not freeze, but becomes glassy at a temperature lower than the bulk freezing point. On rewarming this glassy water there is usually a glass transition visible in a DSC scan. Thus, DSC, as well as NMR techniques can be used to study the states of water in hydrated antifreeze proteins, active fragments or mimetics thereof. In hydrated antifreeze proteins, fragments or mimetics, a fraction of water remains mobile down to very low temperatures. DSC can be used to study the vitrification and crystallization characteristics of water in hydrated antifreeze proteins, active fragments or mimetics thereof. 170 NMR can be used to distinguish characteristically different molecular environments of water molecules in hydrated antifreeze proteins, active fragments or mimetics thereof which can be connected with the binding mechanism of the protein, fragment or mimetic to water molecules. These techniques can also be used to quantify the fraction of “unfrozen water” in the hydrated samples. These studies provide information about the status of the water layers around the antifreeze protein, active fragment or mimetic thereof.

The ability of insect and plant antifreeze proteins to inhibit clathrate hydrate formation was demonstrated by the present inventors in the following examples.

To obtain insect antifreeze protein several systems for expression of the antifreeze protein of Choristoneura fumiferana (CfAFP) were examined. The yeast Pichia pastoris yielded high levels of protein but glycosylated the proteins inappropriately. Transfections of lepidopteran cell lines, also know as High 5 cells, with plasmids containing several different CfAFP isoforms were successful and secreted protein of the predicted size which was not glycosylated. Antifreeze activity was exhibited in the medium of these cells. However, protein levels were low on the order of 5 ng/l of medium. Construction of transgenic Drosophila flies bearing a P-element integrated copy of CfAFP DNA ligated to a secretion sequence resulted in substantial levels of antifreeze activity in the hemolymph of adult flies. Isolation of quantities of AFPs from these small (1 mg) insects, however, may not be commercially practical. Recently new bacterial expression vectors along with compatible bacterial strains have been developed which are specifically designed to fold intransigent proteins. Examples include, but are not limited to, origami lines, various expression plasmids such as pET vectors and various bacterials species. Various cDNAs encoding different CfAFP isoforms have been cloned into these vectors to examine their usefulness in expression of high levels of the AFPs.

Experiments with recombinant CfAFP showed that the insect antifreeze protein retards the growth of the THF hydrate and changes the morphology of the crystal structure.

Hydrate crystals have various morphologies that depend on the crystal structure and external parameters, such as the near-symmetric octahedral and plate-like crystals observed for structure II (Str.II) hydrates. When Str.II hydrates of tetrahydrofuran are grown at the end of a glass pipette at 2.5° C., a single crystal emerges and grows into a regular octahedron. However, if the crystal, while still small, is transferred to a polyvinylpyrrolidone (PVP) containing solution, octahedral crystal growth is inhibited and only plate-like crystal growth is observed (Makogon et al. J. of Cryst. Growth 1997 179: 258-262). Transfer of the crystal to a THF solution containing the hyperactive insect AFP from Choristoneura fumiferama (spruce budworm), CfAFP6, showed similar, slow plate-like growth as seen with PVP. Similarly, transfer of the crystal to a THF solution containing 0.25 mM fish AFP (Type I from winter flounder; wfAFP4) resulted in inhibition of octahedral crystal growth and only plate-like crystal growth was observed. Octahedral crystal habit formation was not inhibited in the presence of control proteins, cytochrome C or serum albumin.

Experiments on the morphology of the inhibited THF crystal showed that slow growth occurs along the <111> face.

Raman experiments also showed the presence of unfrozen water in fish AFP solution and a solution of fish AFP and recombinant CfAFP. DSC experiments for hydrated protein and protein-H₂O solutions also showed the existence of unfrozen water in hydrated Type I fish AFP as well as a solution of fish AFP and recombinant CfAFP. The unfrozen water content in the CfAFP solution was significantly greater than that in fish AFP solution. Also, with CfAFP the melting point was depressed, again even more so than in the fish AFP solutions at a comparable concentration. DSC analysis also revealed a novel exothermic peak that was not seen with control BSA proteins.

In addition to inhibiting growth, both insect antifreeze protein and fish antifreeze protein decreased nucleation rate. This was quantified by timing the stochastic formation of THF hydrate in a multi-sample apparatus. At 0° C., it took less than 5 hours to form crystals in 90% of the samples containing THF solutions, with or without control protein. In contrast, when PVP or wfAFP (both at 0.25 mM) were present, after 5 hours less than 50% of the samples had crystallized. Assuming the phase change in the tested solutions is a one-step transformation (Heneghan et al. J. Chem. Phys. 2001 115: 7599-7608), the nucleation rate, k, and the average lag time, τ, can be derived (see Table 1).

TABLE 1
Nucleation rate (k) and average lag time (τ) of THF
hydrate formation^{a} or the rate, lag time and rate of gas
consumption (r) for propane hydrate^{b}

| | k | τ | r |
|---|---|---|---|
| | (×10⁻³ min⁻¹) | (×10² min) | (KPa/hr.) |
| THF-H₂O | 16.3 ± 2.2 | 0.61 | — |
| THF-H₂O | 56.8 ± 2.1 | 0.18 | — |
| melted | | | |
| PVP 0.25 mM | 3.5 ± 1.1 | 2.86 | — |
| PVP 0.25 mM | 76.6 ± 20.4 | 0.13 | — |
| melted | | | |
| PVP 0.05 mM | 29.6 ± 5.7 | 0.34 | — |
| PVP 0.05 mM | 83.4 ± 16.1 | 0.12 | — |
| melted | | | |
| Cytochrome C | 14.9 ± 1.6 | 0.67 | — |
| 0.25 mM | | | |
| Cytochrome C | 38.8 ± 23.0 | 0.26 | — |
| 0.25 mM | | | |
| melted | | | |
| wfAFP | 1.0 ± 0.2 | 10.31 | — |
| 0.25 mM | | | |
| wfAFP | 0.7 ± 0.2 | 13.89 | — |
| 0.25 mM | | | |
| melted | | | |
| WfAFP | 4.0 ± 0.4 | 2.51 | — |
| 0.05 mM | | | |
| WfAFP | 56.72 ± 5.95 | 0.18 | — |
| 0.05 mM | | | |
| melted | | | |
| CfAFP | 2.9 ± 0.6 | 3.46 | — |
| 0.05 mM | | | |
| CfAFP | 2.7 ± 0.6 | 3.68 | — |
| 0.05 mM | | | |
| melted | | | |
| C₃H₈ + H₂O | 17.9 ± 0.2 | 0.56 | 4.1 ± 0.4 |
| C₃H₈ + H₂O | 58.8 ± 1.2 | 0.17 | 3.7 ± 0.8 |
| (melted) | | | |
| C₃H₈ + H₂O + wfAFP | 5.6 ± 0.1 | 1.79 | 0.8 ± 0.4 |
| 0.25 mM | | | |
| C₃H₈ + H₂O | 5.9 ± 0.1 | 1.70 | 0.7 ± 0.2 |
| (melted) + wfAFP | | | |
| 0.25 mM | | | |

^{a}Measurement recorded at 0° C.
^{b}Measurement recorded at 0° C., starting pressure of propane is 400 KPa.

At 0° C., wfAFP (0.25 mM) decreased the nucleation rate of THF hydrate by a factor of approximately 16, and even when the concentration was reduced 5-fold to 0.05 mM, the rate was still lowered relative to controls by a factor of 4. With a hyperactive insect AFP, CfAFP, at 0.05 mM, the nucleation rate was decreased by a factor of 6; PVP at this concentration did not decrease the nucleation rate of THF hydrate. At a higher PVP concentration (0.25 mM), the rate was approximately 5 times lower than with the THF solution alone, indicating that insect and fish antifreeze proteins are considerably more effective at suppressing hydrate nucleation than the commercial inhibitor.

Hydrate crystallization has a well-recognized “memory effect” in that if hydrates crystallize from a solution and are melted, they can subsequently reform very rapidly. Accordingly, the term “reformation” as used herein is meant to be inclusive of both hydrate crystallization occurring from this memory effect after complete melting and reforming of the same crystals upon re-freezing when melting is incomplete. In pipelines, hydrate crystal reformation can lead to serious blockages after plugs have been decomposed (Sloan, E. D., Jr.: Clathrate Hydrates of Natural Gases. 2^{nd} edition, Marcel Dekker, Inc., New York, USA (1998)). To test the ability of wfAFP to prevent rapid recrystallization, THF hydrates were melted 2° C. above the equilibrium melting point (4.4° C. for THF hydrate), and the time for re-crystallization was determined after the samples were returned to 0° C. Although the formation rate of the second crystallization increased, relative to the initial crystallization, for THF solutions alone, or when a control protein, or when PVP was added, the rate of crystallization did not increase when wfAFP (0.25 mM) was present (see Table 1). An even lower concentration of CfAFP (0.05 mM) was effective at suppressing the nucleation rate after a freeze-thaw cycle. Thus, both insect and fish antifreeze proteins eliminate the memory effect. Accordingly, in this embodiment of the present invention, wherein the rate of clathrate hydrate crystal reformation is decreased and/or the nucleation rate after a freeze-thaw cycle is suppressed and/or the memory effect is inhibited, preferred antifreeze proteins are those containing or forming a β-helix or β-helices, a β-roll, a glycoprotein or globular structure, or an α-helix.

Although the reason for the memory effect is unknown, molecular dynamics simulation on the melting of methane hydrate (Rodger P. M. Ann. N.Y. Acad. Sci. 2000 912: 474-482) suggests that ice- and clathrate-like structures persist in the liquid water when the hydrate decomposes. The inventors herein believe that the memory effect may also arise from impurities that are “imprinted” by the crystallizing hydrate as it nucleates, subsequently becoming efficient nucleating agents for recrystallization. Thus, while not being bound to a particular theory, it is believed that the antifreeze proteins used in the methods and compositions of the present invention may adsorb to the imprinted particles effectively eliminating these nucleators.

To determine if antifreeze proteins inhibit hydrocarbon hydrate formation as well as the model THF clathrate, propane hydrate, another Str.II hydrate was tested. Again, the addition of wfAFP (0.25 mM) increased the time for crystallization at 0° C., decreasing the rate of nucleation by a factor of 3 and hydrate growth rate (in terms of the rate of gas consumption) by a factor of 5, compared to pure propane hydrate. Inhibition of the freezing memory effect of water in propane hydrate mediated by wfAFP was also observed. When the water for propane hydrate synthesis was frozen and then allowed to melt at 2° C. for 1 hour in the presence of the antifreeze protein, the nucleation rate upon re-freezing did not increase. In contrast, water alone showed a characteristic freezing memory effect for propane hydrate formation with a 3-fold increase in the nucleation rate. Further, the hydrate growth rate did not change appreciably, thus indicating that the memory effect is related to the nucleation process but not to crystal growth.

A model for a plant antifreeze protein was also developed. The Lolium perenne (Lm) rye grass (Kuiper et al. Biophysical J. 2001 81: 3560-3566) survives freezing. Its antifreeze protein, LpAFP, has low thermal hysteresis activity, but is very active in the inhibition of ice recrystallization. Using the beta helix structure modeled for the insect antifreeze proteins, the sequence of the plant antifreeze protein was folded into a beta roll, optimized and “docked” to ice using dynamics simulations. Some motifs with some similarity to the “threonine button” ice binding surface of the insect antifreeze proteins were revealed. However, the motifs contained many substitutions which probably account for the low thermal hysteresis activity of the plant antifreeze protein. Further, two multiple-substituted ice binding surfaces appear in the model which may be responsible for the superior ice recrystallization activity.

Thus, these experiments demonstrate the ability of both insect and plant, as well as fish antifreeze proteins to inhibit clathrate hydrate crystal formation and reformation including inhibition of reformation from memory effect and incomplete melting. Further, one of skill in the art can routinely identify active fragments of these proteins as well as mimetics in accordance with the assays described herein which can also be used to inhibit clathrate hydrate crystal formation and reformation. Accordingly, the present invention also provides methods for designing hydrate inhibition chemicals via comparison of such new chemicals to inhibitory activity of antifreeze proteins or active fragments thereof. Preferably, the ability of a new chemical to inhibit clathrate formation or reformation is compared to the ability of an antifreeze protein containing or forming a β helix or β-helices, an α-helix, a β-roll, a glycoprotein, or a globular structure, or an active fragment of the antifreeze protein to inhibit clathrate hydrate formation or reformation. Similar or enhanced ability of the new compound to inhibit clathrate hydrate formation or reformation as compared to the antifreeze protein or active fragment thereof is a indicative of the compound being a hydrate inhibitor.

An antifreeze protein, an active fragment, or a mimetic thereof can be dissolved, preferably in aqueous solution and introduced directly into the solution of the conduit or machinery in which inhibition of clathrate hydrate formation is desired. In a preferred embodiment, the antifreeze protein, active fragment or mimetic thereof is injected into a conduit or machinery in high concentration in aqueous solution so that it is present at an effective concentration to inhibit hydrate formation in the fluid of the conduit or machinery.

In addition, the ability of the antifreeze proteins, active fragments and mimetics thereof to decrease the rate of reformation of clathrate hydrates renders them useful in combination with a second different inhibitor of clathrate hydrate formation. When administered in combination with another clathrate hydrate inhibitor, the antifreeze protein, active fragment or mimetic thereof can be added at a lower concentration as compared to when administered alone as its action is at least additive, and possibly synergistic. Examples of other clathrate hydrate inhibitors which can be administered in combination with an antifreeze protein, active fragment or mimetic thereof include, but are not limited to methanol, glycol and salt.

The following nonlimiting examples are provided to further illustrate the present invention.

### EXAMPLES

### Example 1

### Freezing Point Detected by DSC

Differential scanning calorimetry (DSC) using TA Instruments model TA 2920 modulated DSC was used to measure freezing points using double-distilled water for calibration and sample preparation. HPLC grade tetrahydrofuran samples were sealed in the DSC pan, and cooled from ambient to −40° C. at 5° C./minute. The samples were warmed at 5° C./minute toward room temperature. Five cycles were run for each sample and the averaged freezing point was recorded. THF-H₂O of 1:15 molar ratio was used to prepare Type I fish antifreeze protein (A/F Protein Canada, Inc.) solution and PVP (K30) solution. Two different concentrations of AFP and PVP were used (1.0 mg/ml and 0.2 mg/ml).

### Example 2

### Observations of THF Hydrate Growth

A visual apparatus for observation of THF hydrate growth was constructed (Makogon et al. Crystal Growth 1997 179: 258). It consisted of a transparent cooling jacket with inserted class tube containing a glass pipette. The test solution was placed inside a sample tube and cooled below the hydrate melting point. Then the pipette was placed into the solution and a copper wire cooled with dry ice was inserted into the pipette in order to initiate hydrate formation inside the pipette. The hydrate crystal grew to the edge of the pipette and a small area of the crystal became exposed to the solution in the test tube. Usually the hydrate grew as a single crystal on the tip of the pipette in the test solution.

In these experiments, the THF hydrate crystal was grown in THF-H₂O (1:15 molar ratio) at 2.5° C., and subsequently moved into the THF-H₂O solution (1:15 molar ratio) with AFP or PVP at concentration of 1.0 mg/ml and 0.2 mg/ml, respectively. The continued growth of the crystal was monitored by a HITACHI video camera (HV-62C) equipped with an OPTEM macro video graphics card (ATI TV Wonder VE). THF-H₂O of 1:15 molar ratio was used to prepare solutions containing 1.0 mg/ml or 0.2 mg/ml Type 1 AFP or PVP (K30).

### Example 3

### Induction Time Measurement

For this experiment, seven test tubes with micro-stirring bars, each containing 5 ml of test solution were used. The tubes were immersed in a tank connected to a cooling bath. Thermocouples were used to monitor the sample and determine the onset of hydrate formation, which was indicated by a sudden temperature increase of several degrees. The time difference between the onset point and the time point at which the solution achieved equilibrium was defined as the induction time. For this experiment, the cooling bath was set to either 0.0° or 1.0° C. THF-H₂O of 1:15 molar ratio and AFP-THF-H₂O of 1.0 mg/ml was used. Experiments were run over 24 hours and each sample was assayed three times. If no hydrate formed after 24 hours, the induction time was marked as 24 hours.

1. A method for inhibiting clathrate hydrate plug formation in a conduit or machinery comprising adding to the conduit or machinery an antifreeze protein, an active fragment of the antifreeze protein or a mimetic of the antifreeze protein or active fragment thereof.

2. The method of claim 1 wherein the antifreeze protein contains or forms a β-helix or β-helices, a β-roll, a glycoprotein or a globular structure.

3. The method of claim 1 wherein the antifreeze protein or active fragment is derived from an insect, plant, fungi, protist or bacteria.

4. The method of claim 1 wherein the antifreeze protein or active fragment is derived from an insect or plant.

5. A method for decreasing the rate at which clathrate hydrates reform in a conduit or machinery comprising adding to the conduit or machinery an antifreeze protein, an active fragment of the antifreeze protein or a mimetic of the antifreeze protein or active fragment thereof.

6. The method of claim 5 wherein the antifreeze protein contains or forms a β-helix or β-helices, a β-roll, a glycoprotein, or a globular structure.

7. The method of claim 5 wherein the antifreeze protein or active fragment is derived from an insect, plant, fungi, protist or bacteria.

8. The method of claim 5 wherein the antifreeze protein or active fragment is derived from an insect or plant.

9. The method of claim 5 further comprising addition of a second, different inhibitor of clathrate hydrate formation to the conduit or machinery.

10. The method of claim 1 or 5 wherein the conduit or machinery is a pipeline in oil drilling and exploration or transport.

11. The method of claim 1 or 5 wherein the conduit or machinery is used in a bacterial fermentation process.

12. The method of claim 1 or 5 wherein the conduit or machinery is used in disposal of CO₂ containing waste products.

13. A method for designing and selecting clathrate hydrate inhibitors comprising comparing a test compound's ability to inhibit clathrate formation or reformation to the ability of an antifreeze protein containing or forming a β helix or β-helices, an α-helix, a β-roll, a glycoprotein, or a globular structure, or an active fragment of the antifreeze protein to inhibit clathrate hydrate formation or reformation wherein similar or enhanced ability of the test compound to inhibit clathrate hydrate formation or reformation as compared to the antifreeze protein or active fragment thereof is a indicative of the test compound being a clathrate hydrate inhibitor.

14. A method for inhibiting clathrate hydrate plug formation and/or decreasing the rate of clathrate hydrate plug reformation in the production, storage and transport of hydrocarbons comprising adding to the hydrocarbons an antifreeze protein, an active fragment of the antifreeze protein, or a mimetic of the antifreeze protein or active fragment thereof in an aqueous solution.

15. The method of claim 14 wherein the antifreeze protein contains or forms a β-helix or β-helices, a β-roll, a glycoprotein, or a globular structure.

16. The method of claim 14 wherein the rate of clathrate hydrate plug reformation in the production, storage and transport of hydrocarbons is decreased by inhibition of memory effect.

17. The method of claim 16 wherein the antifreeze protein contains or forms a β-helix or β-helices, a β-roll, a glycoprotein, a globular structure or an α-helix.

18. A composition for inhibiting clathrate hydrate formation or decreasing rate of reformation of clathrate hydrates comprising an antifreeze protein, an active fragment of the antifreeze protein, or a mimetic of the antifreeze protein or active fragment thereof in an aqueous solution.

19. The composition of claim 18 wherein the antifreeze protein contains or forms a β helix or β-helices, a β-roll, a glycoprotein, or a globular structure.

20. The composition of claim 18 further comprising a second, different inhibitor of clathrate hydrate formation.
